(19) United States
(12) Patent Application Publication
(10) Pub. No.: US 2007/0231895 A1
(43) Pub. Date: Oct. 4, 2007

(54) Methods for adapting mammalian cells

(76) Inventors: Gene W. Lee, Chelmsford, MA (US); D. Troy Richards, Spencerport, NY (US); Martin S. Sinacore, Andover, MA (US); Mark Leonard, Manchester, NH (US); Timothy S. Charlebois, Andover, MA (US); Mark Melville, Melrose, MA (US); Robin A. Heller-Harrison, Andover, MA (US)

Correspondence Address: CHOATE, HALL & STEWART LLP/WYETH, PATENT GROUP, TWO INTERNATIONAL PLACE, BOSTON, MA, 02110, US

(21) Appl. No.: 11/591,710

(22) Filed: Nov. 2, 2006

Related U.S. Application Data
(60) Provisional application No. 60/732,818, filed on Nov. 2, 2005.

(51) Int. Cl. C12N 5/06

(52) U.S. Cl. 435/326; 435/375

(57) ABSTRACT

Methods of adapting cells, e.g., mammalian cells, to a cell culture process are provided. When the adapted cells are genetically modified and used for protein production, they exhibit beneficial characteristics, such as being able to attain higher cell densities and/or achieve a higher overall yield of the produced protein.

### CROSS REFERENCE TO RELATED APPLICATIONS

This application is copending with, shares at least one common inventor with, and claims priority to U.S. provisional patent application No. 60/732,818, filed Nov. 2, 2005, the contents of which are hereby incorporated by reference in their entirety.

### BACKGROUND OF THE INVENTION

This disclosure relates generally to methods of adapting mammalian cells, e.g. untransfected mammalian cells, for production of a therapeutic protein of interest. Particularly this disclosure relates to adapting untransfected mammalian cells for superior performance in bioreactors.

Proteins can be produced by using well known recombinant techniques. Transformed cells are commonly cultured in a controlled environment, such as a bioreactor. Most large-scale commercial manufacturing strategies employ suspension cell cultures grown in large stirred-tank reactors. Most cell lines, however, do not readily perform well in high cell density protein production processes (e.g., fed-batch processes) and/or cannot reach the desired high cell densities. Many inhibitors are present or accumulate in the cell culture medium during a production run; these inhibitors may be by-products generated from metabolic processes such as lactate or ammonia, among others. The cell lines are typically grown and maintained initially in conditions that are designed to aid in the propagation and/or survival of these cell lines. Conditions used during protein production (e.g., conditions encountered in production bioreactors), however, can be quite different, with e.g., secondary metabolites and/or other components present and/or accumulated as protein production progresses, which can have potential deleterious effects on the cell line. As non-limiting examples, such deleterious effects may comprise a decrease in the viable cell density and/or a decrease in the final titer, as well as a decrease in the amount and/or quality of the produced protein.

Practicing ordinary methods, numerous experiments are performed to determine whether a cell line has adapted to growth and/or production in a protein production process such as a fed-batch process in a bioreactor. Such experimentation often requires multiple clonal cell lines and/or transfection pools, typically followed by adaptation of the cells to growth in media that will be used in a production bioreactor (e.g., a serum free suspension or a “defined medium”). Once a cell line is adapted, additional rounds of screening and optimization in bench-scale bioreactors are typically performed. Individual clones that exhibit good expression phenotypes in the preliminary research are then typically subjected to additional experimentation to determine which clones also exhibit acceptable growth and/or viability characteristics in a bioreactor, for example a fed-batch bioreactor. Such extensive screening processes are costly and particularly arduous for the practitioner.

Therefore, what is needed are methods for adapting cells to protein production conditions including, but not limited to, bioreactor conditions. What is further needed is a method of adapting untransfected host cells prior to being transformed in such a manner that there are no or minimal detrimental effects on one or more cell culture characteristics (e.g., titer, viability, protein quality, etc.) after transfection and transition into protein production conditions.

### SUMMARY

The present disclosure relates to methods for adapting mammalian cells to a high density protein production process such as, for example, a fed-batch process. Inventive processes can involve both untransfected mammalian cells and genetically manipulated host cells. In certain embodiments, untransfected mammalian cells are adapted to production-matched conditions such as those used during protein production. For example, untransfected mammalian cells may be adapted to a production medium used in a bioreactor during a production run.

Different methods may be employed to adapt the untransfected mammalian cells to production conditions. In certain embodiments, cells are cultured in a production and/or adaptation medium. In certain embodiments, cells are cultured in an adaptation medium with standard iterative splitting cycles performed. For example, subpopulations of the cells may be passaged one or more times, e.g. every three or four days. In certain embodiments, a production and/or adaptation medium generally has higher levels of nutrients, vitamins, and/or trace elements compared to a standard growth medium. In certain embodiments, the cells are then allowed a recovery period between passaging, where the passaged cells are grown in a standard growth medium, e.g. during one of the cycles, before returning the cells to a production and/or adaptation medium.

In certain embodiments, cells are adapted by passaging them in batch-refeed mode, where subpopulations of the cells are split or passaged every seven or eight days. In certain embodiments, passaging the cells after such a longer duration allows an accumulation of secondary metabolites in the cell culture medium. In certain embodiments, adapted cells gain tolerance to and/or begin to take up such secondary metabolites. In certain embodiments, such secondary metabolites comprise inhibitors and/or metabolites that typically accumulate in a bioreactor during a production run, such as, for example, lactate and/or ammonia. In certain embodiments, cells are adapted by growth in a medium that includes one or more inhibitors including, but not limited to, lactate, ammonia, alanine, glutamine, and/or acetolactate. In certain embodiments, such inhibitors and/or metabolites are consistent with inhibitors and/or metabolites typically found in a bioreactor during a production run.

In certain embodiments, untransfected mammalian cells may be adapted by being cultured (and optionally split every three or four days) in a standard growth medium which is supplemented with inhibitors such as alanine, glutamine, acetolactate, ammonia, and lactate. In certain embodiments, cells adapted in such a manner are passaged every three or four days. In certain embodiments, the concentrations of such inhibitors correspond to concentrations typically found in a bioreactor during conditions used for protein production. In certain embodiments, concentrations of some inhibitors include about 2 to about 10 g/L of lactate or about 0.1 to about 0.5 g/L of ammonia, mimicking typical bioreactor conditions. In certain embodiments, cells adapted to bioreactor conditions by one or more methods of the present invention exhibit certain characteristics or phenotypes and/or can develop such phenotypes in which the cells begin to uptake lactate and/or other secondary metabolites, such that the levels of lactate and/or other secondary metabolites actually decrease during one or more time periods during the production run. Such phenotypes may be screened-for because they exhibit qualities desirable in a high-density fed-batch production bioreactor.

In certain embodiments, cells adapted by one or more methods of the present invention are host cells that have not been transfected to produce a protein of interest. In certain embodiments, such adapted host cells are then screened for one or more desirable characteristics. In certain embodiments, once a subpopulation is screened for one or more desirable characteristics, after which the cells may be genetically manipulated (e.g. transfected) to create a cell line that produces a protein of interest. In certain embodiments, such an adapted cell line is placed in a bioreactor where the cell line readily adapts to a high cell density protein production process such as, for example, a fed batch process. In certain embodiments, as a result of the prior adaptation of the untransfected host cells, e.g., mammalian host cells, the genetically manipulated cell line does not have to transition from a standard growth medium to a production medium and/or transitions with fewer and/or less severe deleterious effects. In certain embodiments, prior adaptation minimizes the potential deleterious effects on the cell line, and helps ensure cell line performance and accelerates development timelines. In certain embodiments, such an adapted cell line may uptake secondary metabolites during the protein production run. A person of ordinary skill in the cell culture art can readily determine what components make-up a standard growth medium and a standard production medium. In certain embodiments, growth and protein production conditions differ in the composition of the cell culture media used. During growth and/or protein production phases, conditions of a bioreactor may be altered and/or supplements may be added in order to increase the productivity and/or maintain viability of the cell line. Supplements may include a feed medium and/or one or more additives. Those of ordinary skill in the art will be able to select appropriate media supplements. Further supplements to the mediums will depend on the desired protein product, the parameters (e.g., components, pH, etc.) of the cell culture medium, the methods employed throughout the growth and/or production process, and or any of a variety of other factors known to those of ordinary skill in the art.

In certain embodiments, untransfected mammalian cells are adapted by a method comprising culturing untransfected mammalian cells in an adaptation medium, performing one or more iterative splitting cycles (e.g. every 3 or 4 days) comprising splitting the untransfected mammalian cells in the adaptation medium, allowing a recovery period where the cells are cultured in a standard growth medium, and screening the cells and selecting a subpopulation that exhibits an improved growth and/or viability phenotype compared to an unadapted version of the cells when cultured under conditions of a production bioreactor. In certain embodiments, the cells are transfected with a gene encoding a protein of interest and cultured in a production bioreactor to express the protein of interest. In certain embodiments, an adaptation medium contains increased levels of nutrients, vitamins, and/or trace elements compared to said standard growth medium. In certain embodiments, an adaptation medium contains an increased amount of inhibitory metabolites as compared to a standard production medium prior to cell culture.

In certain embodiments, untransfected mammalian cells are adapted by a method comprising culturing untransfected mammalian cells in an adaptation medium that has been supplemented with side products of primary carbon metabolism, performing one or more iterative splitting cycles comprising splitting the untransfected mammalian cells about every 3 or 4 days in the adaptation medium, allowing a recovery period where the cells are cultured in a standard growth medium, accumulating levels of the side products, and screening the untransfected mammalian cells and selecting a subpopulation that exhibits an improved phenotype compared to an unadapted version of the untransfected mammalian cells when cultured in said the adaptation medium. In certain embodiments, such side products are one or more of lactate, ammonia, alanine, glutamine, and/or acetolactate. In certain embodiments, lactate is initially present at a concentration of about 2 to about 10 g/L. In certain embodiments, ammonia is initially present in a concentration of about 0.1 to about 0.5 g/L. In certain embodiments, the untransfected mammalian cells are adapted to take up said side products of primary carbon sources.

In certain embodiments, untransfected mammalian cells are adapted by a method comprising culturing untransfected mammalian cells for a duration of about 7 or 8 days in a previously-conditioned medium having an accumulation of at least one inhibitory metabolite before splitting the untransfected mammalian cells, and screening the untransfected mammalian cells and selecting a subpopulation that exhibits an improved phenotype in a production bioreactor. In certain embodiments, cells are allowed a recovery period by culturing the cells in a standard growth medium for a duration of about 3 to about 4 days before splitting the cells. In certain embodiments, a conditioned medium contains an accumulation of the inhibitory metabolites through at least one metabolic process of the untransfected mammalian cells. In certain embodiments, such an inhibitory metabolite is one or more of ammonia, alanine, glutamine, acetolactate, and/or lactate. In certain embodiments, such inhibitory metabolites are consistent with those found in a production bioreactor at the end of a typical commercial-scale batch re-feed process.

Any of a variety of suitable culture procedures and/or media (e.g., inoculum media, feed media, etc.) may be used to culture the cells in the process of protein production. Both serum-containing and serum-free media may be used. For example, in certain embodiments, cells are grown in a defined medium. In certain embodiments, cells are grown in a complex medium. In addition, one or more specific culturing methods may be used, altered and/or optimized to culture the cells as appropriate for the specific cell type and protein product. Such procedures are well known and understood by workers and those of ordinary skill within the cell culture art. Other features and advantages of the disclosure will be apparent from the following description of certain embodiments, and from the claims.

### BRIEF DESCRIPTION OF THE DRAWINGS

FIG. 1a shows Seven Day Batch-Refeed Viable Cell Density

FIG. 1b shows Standard Splitting Viable Cell Density

FIG. 1c shows Viable Cell Density

FIG. 2a shows Growth Rate of Monoclonal Antibody Cell Line

FIG. 2b shows Accumulated Integral Viable Cell Density During Fed-Batch

FIG. 3 shows Cell Densities of Cell Cultures Adapted to Lack of Insulin and Control Cell Cultures.

FIG. 4 shows Viability of Cell Cultures Adapted to Lack of Insulin and Control Cell Cultures.

FIG. 5 shows Accumulated IVCD of Cell Cultures Adapted to Lack of Insulin and Control Cell Cultures.

FIG. 6 shows Cell Densities of Cell Cultures Adapted to Lack of Insulin and Control Cell Cultures.

FIG. 7 shows Viability of Cell Cultures Adapted to Lack of Insulin and Control Cell Cultures.

FIG. 8 shows Accumulated IVCD of Cell Cultures Adapted to Lack of Insulin and Control Cell Cultures.

FIG. 9 shows Specific Lactate Consumption Rate of Cell Cultures Adapted to Lack of Insulin and Control Cell Cultures.

### DEFINITIONS

Following long-standing convention, the terms “a” and “an” mean “one or more” when used in this application, including the claims. Even though the invention has been described with a certain degree of particularity, it is evident that many alternatives, modifications, and variations will be apparent to those skilled in the art in light of the disclosure. Accordingly, it is intended that all such alternatives, modifications, and variations, which fall within the spirit and scope of the invention, be embraced by the claims.

The phrase “host cell” refers to cells which are capable of being genetically manipulated and/or are capable of growth and survival in a cell culture medium. Typically, the cells can express a large quantity of an endogenous or heterologous protein of interest and can either retain the protein or secrete it into the cell culture medium.

Host cells are typically “mammalian cells,” which comprise the nonlimiting examples of vertebrate cells, including include baby hamster kidney (BHK), Chinese hamster ovary (CHO), human kidney (293), normal fetal rhesus diploid (FRhL-2), and murine myeloma (e.g., SP2/0 and NS0) cells. One of ordinary skill in the art will be aware of other host cells that may be used in accordance with methods and compositions of the present invention.

The term “cell culture medium” refers to cells in a solution containing nutrients to support cell survival under conditions in which the cells can grow and/or produce a desired protein. The phrase “inoculation medium” or “inoculum medium” refers to a solution or substance containing nutrients in which a culture of cells is initiated. In certain embodiments, a cell culture is supplemented at one or more times with a “feed medium”, with which the cells are fed after initiation of the culture. In certain embodiments, a “Feed medium” contains similar nutrients as the inoculation medium, but is a solution or substance but is a solution or substance with which the cells are fed subsequent to initiation of the culture. In certain embodiments, a feed medium contains one or more components not present in an inoculation medium. In certain embodiments, a feed medium lacks one or more components present in an inoculation medium. A person of ordinary skill in the cell culture art will know without undue experimentation what components make-up such inoculation and feed mediums. Typically, these solutions provide essential and non-essential amino acids, vitamins, energy sources, lipids, and/or trace elements required by a cell for growth and survival.

The term “cell culture characteristic” as used herein refers to an observable and/or measurable characteristic of a cell culture. Methods and compositions of the present invention are advantageously used to improve one or more cell culture characteristics. In certain embodiments, improvement of a cell culture characteristic comprises increasing the magnitude of a cell culture characteristic. In certain embodiments, improvement of a cell culture characteristic comprises decreasing the magnitude of a cell culture characteristic. As non-limiting examples, a cell culture characteristic may be improved growth, increased viability, increased integrated viable cell density, increased titer, and/or increased cell specific productivity. One of ordinary skill in the art will be aware of other cell culture characteristics that may be improved using methods and compositions of the present invention.

The phrase “growth medium” or “standard growth medium” refers to a medium that contains nutrients and supplements that allows cells or a cell line to divide and grow. In certain embodiments, the phrase “production medium” refers to an enriched growth medium that permits high levels of a protein of interest to be expressed. In certain embodiments, a production medium, generally, contains higher levels of nutrients, vitamins, trace elements, and/or other medium components when compared to a standard growth medium. The phrase “adaptation medium” refers to a medium that subjects the cells to protein production conditions that exist in bioreactors (e.g., late-stage production bioreactors), prepares the cells for protein production conditions, and/or minimizes the potentially deleterious effects of transitioning the cells from growth conditions to protein production conditions. In certain embodiments, an adaptation medium includes the presence of one or more secondary metabolites, e.g. those generated from metabolic processes including but not limited to lactate and/or ammonia. In certain embodiments, an adaptation medium includes one or more inhibitors including, but not limited to, lactate, ammonia, alanine, glutamine, and/or acetolactate. In certain embodiments, an adaptation medium comprises a production medium. In certain embodiments, an adaptation medium mimics one or more characteristics of a production medium. In certain embodiments, adapting cells in an adaptation medium results in cells that exhibit decreased or less severe deleterious effects when such adapted cells are switched from a growth or adaptation medium to a production medium. Such adaptation media can be production matched, for example, by the supplementation of production media with such metabolites and/or inhibitors and/or by the accumulation of such metabolites and/or inhibitors in an extended duration cell culture.

The term “defined medium” as used herein refers to a medium in which the composition of the medium is both known and controlled.

The term “complex medium” as used herein refers to a medium contains at least one component whose identity or quantity is either unknown or uncontrolled.

The phrase “cell line” refers to, generally, primary host cells that have been transfected with exogenous DNA, e.g. DNA coding for the desired protein of interest. In certain embodiments, cells derived from the genetically modified cells form the cell line and are placed in a cell culture medium to grow and produce the protein product of interest. In some embodiments, primary host cells are transfected with exogenous DNA coding for a desired protein and/or containing control sequences that activate expression of linked sequences, whether endogenous or heterologous. In certain embodiments, a cell line comprises primary host cells that have been transfected with exogenous DNA and express an heterologous protein of interest. In certain embodiments, a cell line comprises primary host cells that have not been transfected with exogenous DNA and express an endogenous protein of interest.

The “growth phase” of a cell culture medium refers to the period when the cells are undergoing rapid division and growing exponentially, or close to exponentially. Growth phase conditions may include a temperature at about 35° C. to 42° C., generally about 37° C. The length of the growth phase and the culture conditions in the growth phase can vary but are generally known to a person of ordinary skill in the cell culture art. Typically, during the growth phase, cells are grown in a “growth medium” or “standard growth medium”. In certain embodiments, a cell culture medium in a growth phase is supplemented with a feed medium.

The “transition phase” occurs during the period when the cell culture medium is being shifted from conditions consistent with the growth phase to conditions consistent with the production phase. During the transition phase, factors like temperature, among others, are often changed. In certain embodiments, a cell culture medium in a transition phase is supplemented with a feed medium. Methods of the present invention are useful in minimizing the potentially deleterious effect of switching a cell culture from growth phase to production phase conditions.

The “production phase” occurs after both the growth phase and the transition phase. The exponential growth of the cells has ended and protein production is the principal objective. Typically, during the production phase, cells are grown in a production medium. A production medium can be supplemented to initiate production. In certain embodiments, a cell culture medium in a production phase is supplemented with a feed medium. In certain embodiments, the temperature of the cell culture medium during the production phase is lower, generally, than during the growth phase. As is known in the art, in many instances such a decreased temperature facilitates protein production. The production phase continues until a desired endpoint is achieved.

The phrases “spliting”, “passaging”, and “subculturing” refer to the process of dividing a population of cells into two or more subpopulations. For example, a population of cells growing in a cell culture medium may be passaged or subcultured by removing a subpopulation of cells from the cell culture medium and diluting that subpopulation to a lower viable cell density. In certain embodiments, a subpopulation of cells may be diluted in a similar volume with fresh medium. In certain embodiments, the subpopulation of cells is diluted with a cell culture medium that is similar or identical to the cell culture medium in which the original population of cells was growing. For example, if the original population of cells was growing in a production medium, the subpopulation may be diluted with the same or similar production medium. In certain embodiments, the subpopulation of cells is diluted with a cell culture medium that differs from the cell culture medium in which the original population of cells was growing. For example, if the original population of cells was growing in a growth medium a subpopulation of cells may be diluted with a production or adaptation medium. In certain embodiments, the original population of cells has stopped growing (e.g., increasing in cell number) prior to passaging or subculturing a subpopulation. In certain embodiments, a population is passaged two or more times during the adaptation process. For example, a population may be passaged, 2, 3, 4, 5, 6, 7, 8, 9 10, 11, 12, 13, 14, 15, 16, 17, 18, 19, 20 times or more during the adaptation process. Standard practice maintains passaging or “splitting” a cell culture medium every three or four days using a standard growth medium. In certain embodiments, cells are adapted to protein production conditions by growing a population of cells for a longer period of time before passaging. For example, cells may be grown 5, 6, 7, 8, 9, 10, 11, 12, 13, 14 days or more before being passaged.

The phrase “viable cell density” refers to the total number of cells that are surviving in the cell culture medium in a certain volume. The phrase “cell viability” refers to number of cells that are alive compared to the total number of cells, both dead and alive, expressed as a percentage.

“Integrated Viable Cell Density”, “IVCD”: The terms “integrated viable cell density” or “IVCD” as used herein refer to the average density of viable cells over the course of the culture multiplied by the amount of time the culture has run. When the amount of protein produced is proportional to the number of viable cells present over the course of the culture, integrated viable cell density is a useful tool for estimating the amount of protein produced over the course of the culture.

As used herein, the term “antibody” includes a protein comprising at least one, and typically two, VH domains or portions thereof, and/or at least one, and typically two, VL domains or portions thereof. In certain embodiments, the antibody is a tetramer of two heavy immunoglobulin chains and two light immunoglobulin chains, wherein the heavy and light immunoglobulin chains are inter-connected by, e.g., disulfide bonds. The antibodies, or a portion thereof, can be obtained from any origin, including, but not limited to, rodent, primate (e.g., human and non-human primate), camelid, as well as recombinantly produced, e.g., chimeric, humanized, and/or in vitro generated, as described in more detail herein.

Examples of binding fragments encompassed within the term “antigen-binding fragment” of an antibody include, but are not limited to, (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; (vi) a camelid or camelized heavy chain variable domain (VHH); (vii) a single chain Fv (scFv); (viii) a bispecific antibody; and (ix) one or more fragments of an immunoglobulin molecule fused to an Fc region. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-26; Huston et al. (1988) Proc. Natl. Acad. Sci. U.S.A. 85:5879-83). Such single chain antibodies are also intended to be encompassed within the term “antigen-binding fragment” of an antibody. These fragments may be obtained using conventional techniques known to those skilled in the art, and the fragments are evaluated for function in the same manner as are intact antibodies.

The “antigen-binding fragment” can, optionally, further include a moiety that enhances one or more of, e.g., stability, effector cell function or complement fixation. For example, the antigen binding fragment can further include a pegylated moiety, albumin, or a heavy and/or a light chain constant region.

Other than “bispecific” or “bifunctional” antibodies, an antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

The phrases “screen” or “screening” refer to a method of selecting a subpopulation of cells with a certain phenotype that exhibits one or more advantageous characteristics. In certain embodiments, a cell line is screened for one or more cell culture characteristics that are advantageous in a protein production process including, but not limited to improved growth, increased viability, increased integrated viable cell density, increased titer, and/or increased cell specific productivity. In certain embodiments, in a cell line for is screened for one or more cell culture characteristics that are advantageous in a fed-batch bioreactor process, e.g., strong growth and/or viability.

The phrase “bioreactor” refers to a vessel in which a cell culture medium can be contained and internal conditions of which can be controlled during the culturing period, e.g., pH and temperature. One of ordinary skill in the art will be aware of useful and/or appropriate bioreactor conditions that may be controlled, as well as methods of controlling such bioreactor conditions. A “production bioreactor” refers to a bioreactor that is utilized during a protein production process. For example, a production bioreactor may comprise a large commercial-scale vessel from which a large amount of protein may be produced, although a production bioreactor is not limited to such large commercial-scale vessels. In certain embodiments, the volume of a bioreactor is at least 1 liter and may be 10, 100, 250, 500, 1,000, 2,500, 5,000, 8,000, 10,000, 12,000 liters or more, or any volume in between. In addition, bioreactors that may be used include, but are not limited to, a stirred tank bioreactor, fluidized bed reactor, hollow fiber bioreactor, or roller bottle.

The phrase “secondary side-products” and “secondary metabolites” refer to small molecules typically generated as a result of cellular metabolic activity. As is understood by those of ordinary skill in the art, such secondary side-products are often detrimental to cell growth and/or viability. Thus, their minimization or elimination from a cell culture is desirable. Non-limiting examples of secondary side-products include lactate and ammonium ions. In certain embodiments, cells are adapted to protein production conditions by growing the cell in the presence of such secondary side-products. In certain embodiments, cells adapted to protein production conditions exhibit the ability to take up such secondary side-products, such that the levels of secondary side-products decrease over time. One of ordinary skill in the cell culture art will be aware of other metabolic side-products that may be used to adapt cells in accordance with the present invention.

A “fed batch culture” refers to a method of culturing cells in which cells are first inoculated in a bioreactor with an inoculum medium. The cell culture medium is then supplemented at one or more points throughout the production run with a feed medium containing nutritional components and/or other supplements.

A “batch culture” refers to a method of culturing cells in which cells are inoculated in a bioreactor with all the necessary nutrients and supplements for the entirety of the production run. No nutrients, media, etc. are added to the cell culture medium after the cell culture is initiated.

A “perfusion culture” refers to a method of culturing cells that is different from a batch or fed-batch culture method, in which the culture is not terminated, or is not necessarily terminated, prior to isolating and/or purifying an expressed protein of interest, and in which new nutrients and other components are periodically or continuously added to the culture, during which the expressed protein is periodically or continuously harvested. The composition of the added nutrients may be changed during the course of the cell culture, depending on the needs of the cells, the requirements for optimal protein production, and/or any of a variety of other factors known to those of ordinary skill in the art.

The phrase “batch-refeed” refers to a mode of operating a bioreactor or a method of passaging cells. In certain embodiments, a batch-refeed process comprises passaging cells every 7 or 8 days compared to other modes in bioreactors where cells are not passaged or passaged less frequently. On a smaller scale employing standard splitting methods, cells are generally passaged sooner, e.g. every 3 or 4 days, compared to batch-refeed. In certain embodiments, batch-refeed methods are used to adapt a cell culture such that it is able to achieve an improved cell culture characteristic including, but not limited to, an improved growth rate, increased viability, increased integrated viable cell density, increased titer, and/or increased cell specific productivity In certain embodiments, a batch-refeed process utilizes a more enriched culture and/or a medium that contains secondary metabolites and/or other inhibitors in order to adapt cells to protein production conditions. The phrase “secondary metabolites” refers to by-products of metabolic processes of cell functions.

The phrase “expression” refers to the transcription and the translation that occurs within a host cell. The level of expression relates, generally, to the amount of protein being produced by the host cell.

The phrase “protein” or “protein product” refers to one or more chains of amino acids. As used herein, the term “protein” is synonymous with “polypeptide” and, as is generally understood in the art, refers to at least one chain of amino acids liked via sequential peptide bonds. In certain embodiments, a “protein of interest” is a protein encoded by an exogenous nucleic acid molecule that has been transformed into a host cell. In certain embodiments, where the “protein of interest” is encoded by an exogenous DNA with which the host cell has been transformed, the nucleic acid sequence of the exogenous DNA determines the sequence of amino acids. In certain embodiments, a “protein of interest” is a protein encoded by a nucleic acid molecule that is endogenous to the host cell. In certain embodiments, expression of such an endogenous protein of interest is altered by transfecting a host cell with an exogenous nucleic acid molecule that may, for example, contain one or more regulatory sequences and/or encode a protein that enhances expression of the protein of interest. Methods and compositions of the present invention may be used to produce any protein of interest, including, but not limited to proteins having pharmaceutical, diagnostic, agricultural, and/or any of a variety of other properties that are useful in commercial, experimental and/or other applications. In addition, a protein of interest can be a protein therapeutic. Namely, a protein therapeutic is a protein that has a biological effect on a region in the body on which it acts or on a region of the body on which it remotely acts via intermediates. Examples of protein therapeutics are discussed in more detail below. In certain embodiments, proteins produced using methods and/or compositions of the present invention may be processed and/or modified. For example, a protein to be produced in accordance with the present invention may be glycosylated.

The term “titer” as used herein refers to the total amount of recombinantly expressed protein produced by a cell culture in a given amount of medium volume. Titer is typically expressed in units of milligrams or micrograms of protein per milliliter of medium.

One of skill in the art will recognize that the methods disclosed herein may be used to culture many of the well-known mammalian cells routinely used and cultured in the art, i.e., the methods disclosed herein are not limited to use with only the instant disclosure.

### DETAILED DESCRIPTION OF CERTAIN EMBODIMENTS OF THE INVENTION

It has been discovered that mammalian cells, e.g. untransfected mammalian cells, including, e.g., Chinese hamster ovary cells, can be adapted to environments consistent with production bioreactor conditions to improve cell growth during subsequent production of a protein of interest. Such methods are also applicable to both untransfected mammalian cells and to transfected mammalian cells (e.g., mammalian cells transfected with a cDNA or genomic construct causing the expression, as from an expression vector, of a desired recombinant protein). The present invention may be used to adapt cells for the advantageous production of any therapeutic protein, such as, for example, pharmaceutically or commercially relevant enzymes, receptors, antibodies (e.g., monoclonal and/or polyclonal antibodies), Fc fusion proteins, cytokines, hormones, regulatory factors, growth factors, coagulation/clotting factors, antigen binding agents, etc. One of ordinary skill in the art will be aware of other proteins that can be produced in accordance with the present invention, and will be able to use methods disclosed herein to produce such proteins.

Methods of the present invention for adapting untransfected mammalian cells have the potential for identifying cell lines that yield superior productivities and/or exhibit superior protein production characteristics. Furthermore, methods of the present invention may help in the identification of suitable candidate cell lines more quickly and with less effort as compared to standard cell line development procedures. For example, standard processes for identifying cell line candidates typically require numerous experiments on different scales and additional testing for robustness.

Host cells, prior to being transformed are considered untransfected. Traditionally, when creating a new cell line for development, untransfected host cells were initially transfected, or transformed, with exogenous DNA to express of a protein of interest. Using such prior methods, the host cells were not generally experimented with or altered prior to transfection; experimentation and research began on cell line development after the host cells were genetically manipulated to produce a protein product.

In certain embodiments, untransfected mammalian cells can be adapted to protein production conditions with improved performance in batch, fed-batch, and/or perfusion bioreactor processes according to one or more methods of the present invention. In certain embodiments, such adapted cells are capable of maintaining an improved phenotype, for example exhibiting stronger growth, increased viability, increased integrated viable cell density, increased titer, increased cell specific productivity and/or higher cell densities through such development. Certain embodiments of inventive methods described herein may be employed to adapt untransfected mammalian cells to a batch, fed-batch, and/or perfusion protein production process having a superior performance.

Any of a variety of commercially available media such as, for example, Minimal Essential Medium (MEM, Sigma), Ham's F10 (Sigma), or Dulbecco's Modified Eagle's Medium (DMEM, Sigma) may be used as the base medium. Such base media may then be supplemented with amino acids, vitamins, inorganic salts, trace elements, and/or other components to produce growth and/or production mediums. In certain embodiments, cells may be adapted by culturing transfected or untransfected mammalian cells in a production medium similar or identical to a production medium. In certain embodiments, cells are adapted by growing the cells under conditions similar or identical to conditions typically encountered under production conditions in a bioreactor (e.g., in a medium consistent with the medium typically found in a bioreactor) operated in batch mode, fed-batch mode, and/or perfusion mode. In certain embodiments, cells to be adapted are untransfected. In certain embodiments, cells to be adapted have been transfected with an exogenous nucleic acid molecule, for example a nucleic acid molecule that expresses a protein of interest. In certain embodiments, one or more cell culture characteristics is improved during a protein production phase by adapting cells to protein production conditions prior to transfection with an exogenous nucleic acid molecule. In certain embodiments, such an improved cell culture characteristic includes, without limitation, improved growth, improved the overall cell viability, increased integrated viable cell density, increased titer, and/or increased cell specific productivity. A production medium is typically more enriched than a growth medium and is, for example, supplemented with higher levels of nutrients, vitamins, trace elements, and/or other media components compared to a growth medium.

It is typical practice in the art for cells to be continuously cultured in growth medium during cell line development and not encounter production medium until the start of a fed-batch production assay. In certain embodiments, adaptation of untransfected mammalian cells in a protein production medium and/or under protein production conditions prior to transfection, rather than growth medium and/or growth conditions, minimizes the potentially deleterious effects of transitioning the cells from one environment to another, e.g., from growth conditions to protein production conditions post-transfection.

In certain embodiments, methods of the present invention are useful for generating a host cells line that is adapted to protein production conditions. Such an adapted host cell line is capable of being transfected with any of a variety of proteins of interest. In certain embodiments, such an adapted, transfected cell line is placed directly into protein production conditions. In certain embodiments, such an adapted, transfected cell line is grown to a desired cell density and/or a desired cell number during a growth phase, after which the cell line is transitioned into a protein production phase. According to the present invention, such an adapted, transfected cell line exhibits fewer and less severe deleterious effects during the transition phase compared to an non-adapted, transfected cell line.

In certain embodiments, in order to produce a protein of interest, adapted host cells are transfected with an exogenous nucleic acid molecule. In certain embodiments, a nucleic acid molecule introduced into the cell encodes the protein desired to be expressed according to the present invention. In certain embodiments, a nucleic acid molecule contains a regulatory sequence or encodes a gene product that induces or enhances the expression of the desired protein by the cell. As a non-limiting example, such a gene product may be a transcription factor that increases expression of the protein of interest.

In certain embodiments, a nucleic acid that directs expression of a protein is stably introduced into the host cell. In certain embodiments, a nucleic acid that directs expression of a protein is transiently introduced into the host cell. One of ordinary skill in the art will be able to choose whether to stably or transiently introduce the nucleic acid into the cell based on experimental, commercial or other needs.

A gene encoding a protein of interest may optionally be linked to one or more regulatory genetic control elements. In some embodiments, a genetic control element directs constitutive expression of the protein. In some embodiments, a genetic control element that provides inducible expression of a gene encoding the protein of interest can be used. Use of an inducible genetic control element (e.g., an inducible promoter) allows for modulation of the production of the protein in the cell. Non-limiting examples of potentially useful inducible genetic control elements for use in eukaryotic cells include hormone-regulated elements (see e.g., Mader, S, and White, J. H., Proc. Natl. Acad. Sci. USA 90:5603-5607, 1993), synthetic ligand-regulated elements (see, e.g. Spencer, D. M. et al., Science 262:1019-1024, 1993) and ionizing radiation-regulated elements (see e.g., Manome, Y. et al., Biochemistry 32:10607-10613, 1993; Datta, R. et al., Proc. Natl. Acad. Sci. USA 89:10149-10153, 1992). Additional cell-specific or other regulatory systems known in the art may be used in accordance with methods and compositions described herein.

Any protein that is expressible in a host cell may be produced in accordance with methods and compositions of the present invention. The protein may be expressed from a gene that is endogenous to the host cell, or from a heterologous gene that is introduced into the host cell. The protein may be one that occurs in nature, or may alternatively have a sequence that was engineered or selected by the hand of man. A protein to be produced may be assembled from protein fragments that individually occur in nature. Additionally or alternatively, the engineered protein may include one or more fragments that are not naturally occurring.

Any host cell susceptible to cell culture, and to expression of proteins, may be utilized in accordance with the present invention. In certain embodiments, host cells are mammalian cells, such as, for example, Chinese hamster ovary (CHO) cells. Other non-limiting examples of mammalian cells that may be used in accordance with the present invention include BALB/c mouse myeloma line (NSO/I, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells+/−DHFR(CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci., 383:44-68 (1982)); MRC 5 cells; FS4 cells; and a human hepatoma line (Hep G2).

In certain embodiments, cells (e.g. untransfected mammalian cells) are adapted by culturing cells in growth medium supplemented with inhibitors. In certain embodiments, such inhibitors include inhibitors that are typically found when cells are grown under protein production conditions. Non-limiting examples of such inhibitors include lactate, ammonia, alanine, glutamine, and/or acetolactate. One of ordinary skill in the art will be aware of other inhibitors that may be used in accordance with the present invention to adapt cells to protein production conditions.

In certain embodiments, cells (e.g. untransfected mammalian cells) are adapted by culturing cells in an adaptation medium that lacks or comprises a reduced concentration of one or more components traditionally found in production media. For example, traditional production media typically contain insulin at a concentration of about 10 mg/L or greater. Thus, in certain embodiments, cells are adapted by culturing cells in an adaptation medium that lacks insulin. In certain embodiments, cells are adapted by culturing cells in an adaptation medium that contains insulin at a concentration lower than an insulin concentration traditionally found in production media. One of ordinary skill in the art will be aware of other components that are typically present in production media, and will be able to use methods of the present invention to adapt cells to media lacking or comprising a reduced concentration such components.

In certain embodiments, cells are adapted by culturing (e.g., continuous culturing) of untransfected mammalian cells in growth medium supplemented with one or more inhibitors. Non-limiting examples of such inhibitors include secondary side-products of primary carbon sources. For example, some secondary side-products include, but are not limited to, lactate, alanine, glutamine, acetolactate, and/or ammonia. In certain embodiments, the concentrations of such secondary side-products present in and/or added to an adaptation medium mimic those typically encountered in bioreactor conditions, such as, for example, about 2.0 to about 10.0 g/L of lactate and/or about 0.1 to about 0.5 g/L of ammonia. In certain embodiments, cells are adapted under conditions in which the concentration of lactate in the adaptation medium is about 1, 2, 3, 4, 5, 6, 7, 8, 9, 10, 11, 12, 13, 14, 15 g/L or higher. Additionally or alternatively, in certain embodiments, cells are adapted under conditions in which the concentration of ammonia in the adaptation medium is about 0.1, 0.2, 0.3, 0.4, 0.5, 0.6, 0.7, 0.8, 0.9, 1.0, 1.1, 1.2, 1.3, 1.4, 1.5 g/L or higher.

In certain embodiments, individual untransfected mammalian cells and/or subpopulations of untransfected mammalian cells that have been adapted to protein production conditions according to one or more methods of the present invention may exhibit phenotypes consistent with the uptake of such secondary side-products, such that levels of secondary side products actually decrease over time. n certain embodiments, such adapted untransfected mammalian cells retain the ability to take up secondary side-products after they are transfected with an exogenous nucleic acid molecule to produce a protein of interest. In certain embodiments, such adapted, transfected mammalian cells generally perform better in a subsequent protein production process (e.g. a fed-batch bioreactor process) relative to untransfected mammalian cells that do not uptake the side-products and/or that have not been adapted to uptake such secondary side-products. For example, such adapted, transfected mammalian cells perform better in a subsequent batch, fed-batch and/or perfusion protein production processes. In a production reactor (e.g., a fed-batch production reactor), such secondary side-products may often accumulate to levels that are generally inhibitory to further cell growth and/or to viability. In certain embodiments, subpopulations of untransfected mammalian cells that have been adapted according to methods of the present invention grow in a supplemented medium can grow well under the inhibitory and stressful conditions typically encountered in a production bioreactor.

As is known in the cell culture art, in many instances the temperature of a cell culture is decreased the cell culture is switched from growth conditions to protein production conditions. In certain embodiments, cells are adapted by culturing the cells at a temperature conducive to the production of a protein product. In certain embodiments, cells are adapted by culturing them at a temperature of about 31° C., although methods of the present invention are not limited to such a temperature. For example, cells may be adapted by culturing them at a temperature of about 20, 21, 22, 23, 24, 25, 26, 27, 28, 29, 30, 31, 32, 33, 34, 35, 36, 37, 38, 39, 40, 41, 42, 43, 44, or 45° C. One of ordinary skill in the art will be aware of temperature(s) suitable for protein production, which temperature may depend, at least in part, on the cell line, the protein to be produced, other culture conditions, and/or any other factor deemed to be important by those of ordinary skill in the art.

In certain embodiments, cells are adapted by continuous culturing of untransfected mammalian cells for a longer duration in a “batch-refeed” mode. In certain embodiments, untransfected cells are adapted by such “batch-refeed” adaptation methods. In certain embodiments, transfected cells are adapted by such “batch-refeed” adaptation methods. Typical cell culture operations may employ a cell culture management regimen of splitting, passaging, or sub-culturing a cell culture every three or four days. In certain embodiments of “batch-refeed” adaptation methods, untransfected mammalian cells are cultured for longer durations, such as seven or eight days, before being sub-cultured. In certain embodiments, mammalian cells are cultured for 5, 6, 7, 8, 9, 10, 11, 12, 13, 14 days or more before being sub-cultured. Such longer duration cultures have the dual effect of adapting cells for protein production conditions by increasing cell density and/or by accumulating higher levels of secondary metabolites in the conditioned medium to which the cells can adapt.

In certain embodiments, untransfected mammalian cells may be continuously cultured under “production-matched” conditions. In certain embodiments, untransfected cells may be cultured with iterative cycles of cell culture under production-matched conditions followed by passaging subpopulations of the cells after 3 or 4 days under “recovery” or standard growth conditions with the option of using a growth medium. In certain embodiments, a population is passaged multiple times during the adaptation process. For example, a population may be passaged, 2, 3, 4, 5, 6, 7, 8, 9, 10, 11, 12, 13, 14, 15, 16, 17, 18, 19, 20 times or more during the adaptation process. In certain embodiments, cells are adapted to protein production conditions by growing a population of cells for a longer period of time before passaging. For example, cells may be grown 5, 6, 7, 8, 9, 10, 11, 12, 13, 14 days or more before being passaged. Such passaged subpopulations may be screened for one or more desirable characteristics. In certain embodiments, after several cycles of iterative or continuous adaptation, subpopulations of cells will emerge that exhibit superior characteristics including, for example, improved growth and/or viability relative to the unadapted starting population, in the production-matched conditions. In addition, in certain embodiments, certain subpopulations will begin to take up side products, e.g. lactate, such that the level of that side product decreases over time, enhancing the performance of that cell line in a production bioreactor. In certain embodiments, one or more screened subpopulations of cells that exhibit one or more desirable characteristics (e.g., strong growth) are then transformed such that they produce a protein of interest, after which the transfected subpopulations are placed into protein production conditions (e.g., conditions consistent with those to which the cells have been adapted).

In certain embodiments, cells are grown in accordance with any of the cell culture methods described in U.S. patent application Ser. Nos. 11/213,308,11/213,317 and 11/213,633 each of which was filed Aug. 25, 2005, and each of which is herein incorporated by reference in its entirety. For example, in certain embodiments, the cells may be grown in a culture medium in which the cumulative amino acid concentration is greater than about 70 mM. In certain embodiments, the cells may be grown in a culture medium in which the molar cumulative glutamine to cumulative asparagine ratio is less than about 2. In certain embodiments, the cells may be grown in a culture medium in which the molar cumulative glutamine to cumulative total amino acid ratio is less than about 0.2. In certain embodiments, the cells may be grown in a culture medium in which the molar cumulative inorganic ion to cumulative total amino acid ratio is between about 0.4 to 1. In certain embodiments, the cells may be grown in a culture medium in which the combined cumulative glutamine and cumulative asparagine concentration is between about 16 and 36 mM. In certain embodiments, the cells may be grown in a culture medium that contains two, three, four or all five of the preceding medium conditions. Use of such media allows high levels of protein production and lessens accumulation of certain undesirable factors such as ammonium and/or lactate.

In some embodiments, the cells are grown under one or more of the conditions described in U.S. Provisional Patent Application Ser. No. 60/830,658, filed Jul. 13, 2006 and incorporated herein by reference in its entirety. For example, in some embodiments, cells are grown in a culture medium that contains manganese at a concentration between approximately 10 and 600 nM. In some embodiments, cells are grown in a culture medium that contains manganese at a concentration between approximately 20 and 100 nM. In some embodiments, cells are grown in a culture medium that contains manganese at a concentration of approximately 40 nM. Use of such media in growing glycoproteins results in production of a glycoprotein with an improved glycosylation pattern (e.g. a greater number of covalently linked sugar residues in one or more oligosaccharide chains).

Components and/or supplements of the production medium and growth medium can be readily determined by one skilled in the cell culture art. As is known in the art, components and/or supplements may vary depending on the host cell used and the desired protein of interest. In addition, the conditions and amount of side-products produced may or will vary with different bioreactor conditions and with each different cell line. The present disclosure is further illustrated by the following, non-limiting examples. Any modifications that might become necessary in the course of the adaptation of mammalian cells to cell culture medium for production of different proteins are well within the art of cell culture.

### EXAMPLES

### Example 1

### Adaptation of Untransfected Chinese Hamster Ovary Cells to Production Matched Conditions

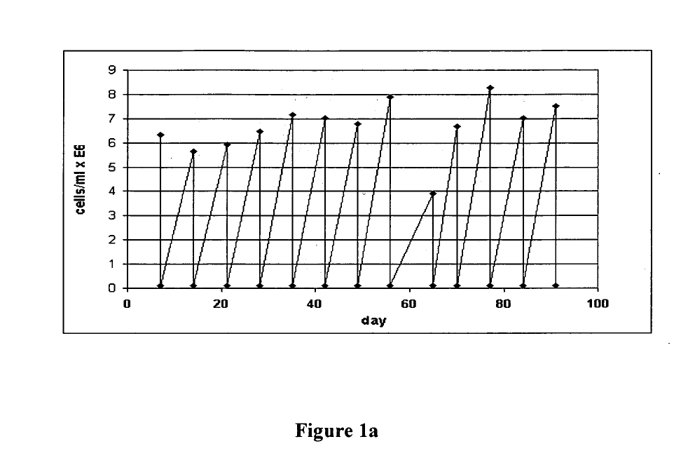

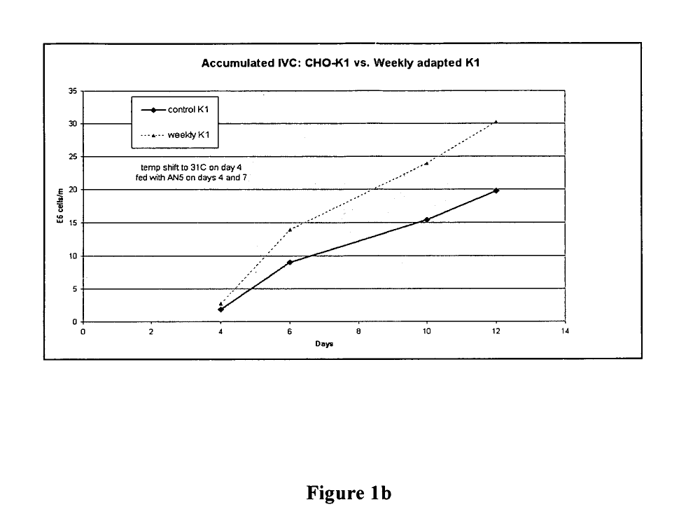

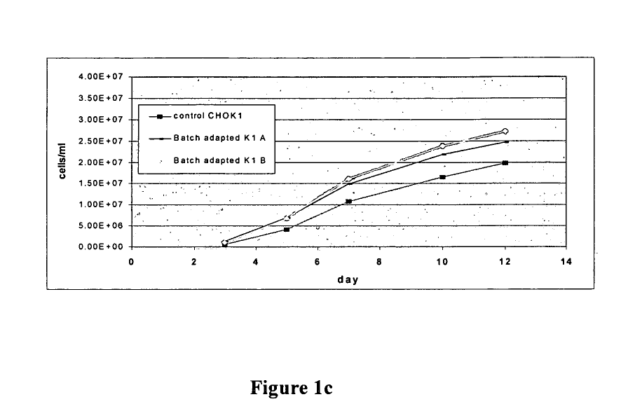

Untransfected CHOK1 cells were cultured either in standard 3 day/4 day batch-refeed conditions in growth medium, components listed in Table 1 below, or cultured in a 7-day batch-refeed mode in enriched production medium, components listed in Table 2 below, for multiple cycles. The cells were cultured at 37° C. in a working volume from 10 to about 30 ml. The cell numbers from the experiment are shown in FIGS. 1a and 1b. At around day 58 of the batch-refeed method of FIG. 1a, the cells were passaged for 9 days, rather than 7, and the subsequent passaged did not grow well. After the poor 7-day passage, however, the cells were able to recover.

TABLE 1

| Component | mg/L |
|---|---|
| alanine | 17.80 |
| arginine | 347.97 |
| asparagine•H2O | 75.00 |
| aspartic acid | 26.20 |
| cysteine•HCl•H2O | 70.19 |
| cystine•2HCl | 62.25 |
| glutamic acid | 29.40 |
| monosodium glutamate | 0.00 |
| glutamine | 1163.95 |
| glycine | 30.00 |
| histidine•HCl•H2O | 46.00 |
| isoleucine | 104.99 |
| leucine | 104.99 |
| lysine•HCl | 145.99 |
| methionine | 29.80 |
| phenylalanine | 65.99 |
| proline | 68.99 |
| serine | 126.00 |
| threonine | 94.99 |
| tryptophan | 16.00 |
| tyrosine•2Na•2H2O | 103.79 |
| valine | 93.99 |
| biotin | 0.20 |
| calcium pantothenate | 2.24 |
| choline chloride | 8.98 |
| folic acid | 2.65 |
| inositol | 12.60 |
| nicotinamide | 2.02 |
| pyridoxal•HCl | 2.00 |
| pyridoxine•HCl | 0.03 |
| riboflavin | 0.22 |
| thiamine•HCl | 2.17 |
| vitamin B12 | 0.78 |
| CaCl2 | 116.09 |
| KCl | 311.77 |
| Na2HPO4 | 70.99 |
| NaCl | 5539.00 |
| NaH2PO4•H2O | 62.49 |
| MgSO4 | 48.83 |
| MgCl2 | 28.61 |
| NaHCO3 | 2440.00 |
| Sodium Selenite | 0.005 |
| Fe(NO3)3•9H2O | 0.050 |
| CuSO4 | 0.001 |
| FeSO4•7H2O | 0.840 |
| ZnSO4•7H2O | 0.430 |
| Hydrocortisone | 0.036 |
| Putrescine•2HCl | 1.080 |
| linoleic acid | 0.040 |
| thioctic acid | 0.100 |
| D-glucose (Dextrose) | 6150.7 |
| PVA | 2400.000 |
| Nucellin | 10.000 |
| Sodium Pyruvate | 54.995 |

TABLE 2

| Components | mg/L |
|---|---|
| alanine | 24.87 |
| arginine | 423.43 |
| asparagine•H2O | 173.90 |
| aspartic acid | 52.72 |
| cysteine•HCl•H2O | 70.01 |
| cystine•2HCl | 62.09 |
| glutamic acid | 41.08 |
| monosodium glutamate | 0.00 |
| glutamine | 1162.40 |
| glycine | 35.92 |
| histidine•HCl•H2O | 75.27 |
| isoleucine | 151.90 |
| leucine | 172.69 |
| lysine•HCl | 218.38 |
| methionine | 53.55 |
| phenylalanine | 98.81 |
| proline | 96.40 |
| serine | 273.07 |
| threonine | 132.81 |
| tryptophan | 28.99 |
| tyrosine•2Na•2H2O | 145.10 |
| valine | 131.17 |
| biotin | 0.36 |
| calcium pantothenate | 4.03 |
| choline chloride | 16.11 |
| folic acid | 4.76 |
| inositol | 22.64 |
| nicotinamide | 3.61 |
| pyridoxal•HCl | 1.99 |
| pyridoxine•HCl | 1.67 |
| riboflavin | 0.40 |
| thiamine•HCl | 3.92 |
| vitamin B12 | 1.34 |
| CaCl2 | 115.8 |
| KCl | 310.9 |
| Na2HPO4 | 70.8 |
| NaCl | 3705.0 |
| NaH2PO4•H2O | 114.3 |
| MgSO4 | 48.7 |
| MgSO4•7H2O | 8.6 |
| MgCl2 | 28.5 |
| NaHCO3 | 2440.0 |
| Sodium Selenite | 0.007 |
| Fe(NO3)3•9H2O | 0.050 |
| CuSO4 | 0.001 |
| CuSO4•5H2O | 0.007 |
| FeSO4•7H2O | 1.543 |
| ZnSO4•7H2O | 1.384 |
| MnSO4*H2O | 0.0002 |
| Na2SiO3*9H2O | 0.140 |
| (NH4)6Mo7O24*4H2O | 0.001 |
| NH4VO3 | 0.001 |
| Hydrocortisone | 0.086 |
| Putrescine•2HCl | 2.480 |
| linoleic acid | 0.057 |
| thioctic acid | 0.142 |
| D-glucose (Dextrose) | 11042.2 |
| PVA | 2520.0 |
| Nucellin | 14.000 |
| Sodium Pyruvate | 54.849 |

Cells were then evaluated in a fed-batch production assay, where the working volume was between 10 and 30 ml. The cells were cultured at 37° C. for four days and then the temperature was shifted to 31° C. until day 12, when the assay completed. The cell density and viability were compared and the results are illustrated in FIG. 1c. Cells cultured in 7-day batch-refeed mode exhibited higher cell densities than cells cultured in standard 3-day/4 day batch refeed mode. Similarly, two different groups of cells that were adapted for growth in production medium, components listed in Table 3 below, achieved higher cell densities than the unadapted control starting population.

TABLE 3

| Component | mg/L |
|---|---|
| alanine | 17.86 |
| arginine | 698.24 |
| asparagine•H2O | 3000.00 |
| aspartic acid | 220.16 |
| cysteine•HCl•H2O | 70.63 |
| cystine•2HCl | 468.75 |
| monosodium glutamate | 33.91 |
| glutamine | 584.00 |
| glycine | 115.87 |
| histidine•HCl•H2O | 476.13 |
| isoleucine | 572.57 |
| leucine | 1034.01 |
| lysine•HCl | 1405.91 |
| methionine | 388.65 |
| phenylalanine | 508.63 |
| proline | 541.23 |
| serine | 1055.38 |
| threonine | 566.62 |
| tryptophan | 275.04 |
| tyrosine•2Na•2H2O | 746.00 |
| valine | 751.41 |
| biotin | 2.69 |
| calcium pantothenate | 22.00 |
| choline chloride | 158.97 |
| folic acid | 26.01 |
| inositol | 164.51 |
| nicotinamide | 26.31 |
| pyridoxal•HCl | 2.04 |
| pyridoxine•HCl | 36.24 |
| riboflavin | 2.42 |
| thiamine•HCl | 39.56 |
| vitamin B12 | 21.24 |
| CaCl2 | 116.92 |
| KCl | 313.91 |
| KH2PO4 | 0.00 |
| Na2HPO4 | 56.78 |
| NaH2PO4•H2O | 647.92 |
| MgSO4 | 138.44 |
| MgCl2 | 28.59 |
| NaHCO3 | 2000.00 |
| Sodium Selenite | 0.069 |
| Fe(NO3)3•9H2O | 0.050 |
| CuSO4 | 0.010 |
| CuSO4•5H2O | 0.100 |
| FeSO4•7H2O | 4.170 |
| ZnSO4•7H2O | 2.649 |
| MnSO4*H2O | 0.034 |
| Na2SiO3*9H2O | 0.284 |
| (NH4)6Mo7O24*4H2O | 0.247 |
| NH4VO3 | 0.002 |
| NiSO4*6H2O | 0.005 |
| SnCl2*2H2O | 0.001 |
| AlCl3*6H2O | 0.001 |
| CrCl3 | 0.016 |
| Kl | 0.033 |
| H3BO3 | 0.012 |
| Hydrocortisone | 0.540 |
| Putrescine•2HCl | 15.000 |
| linoleic acid | 0.291 |
| thioctic acid | 0.718 |
| D-glucose (Dextrose) | 15016.08 |
| PVA | 2560.00 |
| Nucellin | 50.00 |
| Sodium Pyruvate | 55.18 |

### Example 2

### Adaptation of Transfected Product Cell Line to Production-matched Conditions

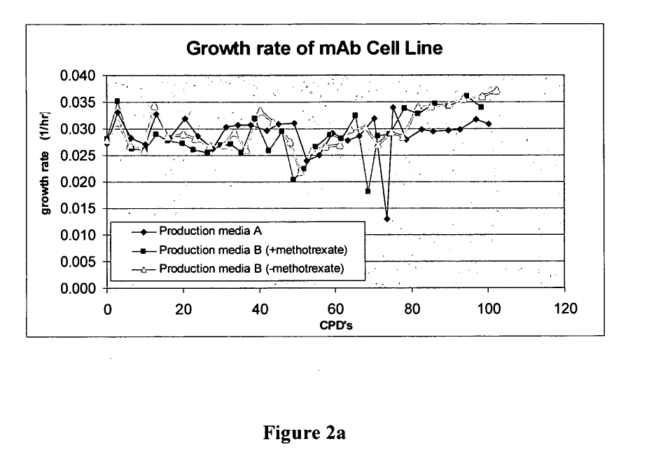

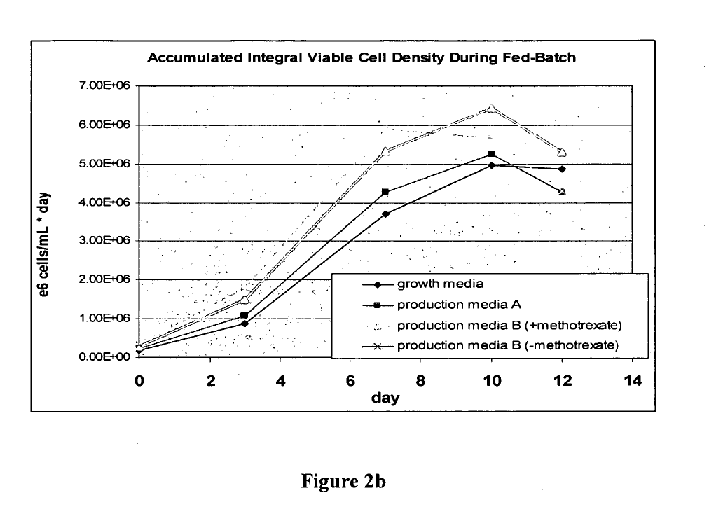

A cell line expressing the heavy and light chain genes of a monoclonal antibody was cultured under standard 3 day/4 day batch refeed conditions, using either standard growth medium, components listed in Table 1, or two versions of production medium, “A” and “B”. Production medium A, components listed in Table 2, was moderately enriched relative to growth medium, and production medium B, components listed in Table 4 below, was significantly enriched relative to growth medium. The effects of methotrexate (MTX) were also tested in production medium “B”, 1.5 μM of MTX was added into one culture with production medium “B” and not into another. The cells were cultured at 37° C. in a working volume from 10 to about 30 ml. Growth rates during adaptation in these media are shown in FIG. 2a.

TABLE 4

| Components | mg/L |
|---|---|
| alanine | 5.95 |
| arginine | 232.75 |
| asparagine•H₂O | 750.00 |
| aspartic acid | 73.39 |
| cysteine•HCl•H₂O | 23.54 |
| cystine•2HCl | 250.00 |
| glutamic acid | 0.00 |
| monosodium glutamate | 44.30 |
| glutamine | 1160.00 |
| glycine | 38.62 |
| histidine•HCl•H₂O | 158.71 |
| isoleucine | 190.86 |
| leucine | 344.67 |
| lysine•HCl | 468.64 |
| methionine | 129.55 |
| phenylalanine | 169.54 |
| proline | 180.41 |
| serine | 351.79 |
| threonine | 188.87 |
| tryptophan | 91.68 |
| tyrosine•2Na•2H₂O | 250.00 |
| valine | 250.47 |
| biotin | 0.90 |
| calcium pantothenate | 7.33 |
| choline chloride | 52.99 |
| folic acid | 8.67 |
| inositol | 54.84 |
| nicotinamide | 8.77 |
| pyridoxal•HCl | 0.68 |
| pyridoxine•HCl | 12.08 |
| riboflavin | 0.81 |
| thiamine•HCl | 13.19 |
| vitamin B12 | 7.08 |
| CaCl₂ | 109.0 |
| KCl | 604.6 |
| Na₂HPO₄ | 18.9 |
| NaCl | 2000.0 |
| NaH₂PO₄•H₂O | 216.0 |
| MgSO₄ | 46.1 |
| MgSO₄•7H₂O | 80.0 |
| MgCl₂ | 9.5 |
| NaHCO₃ | 3300.0 |
| Sodium Selenite | 0.035 |
| Fe(NO₃)₃•9H₂O | 0.017 |
| CuSO₄ | 0.003 |
| CuSO₄•5H₂O | 0.050 |
| FeSO₄•7H₂O | 2.500 |
| ZnSO₄•7H₂O | 0.883 |
| MnSO4*H2O | 0.0169 |
| Na2SiO3*9H2O | 0.142 |
| (NH4)6Mo7O24*4H2O | 0.124 |
| NH4VO3 | 0.001 |
| NiSO4*6H2O | 0.003 |
| CrCl3 | 0.008 |
| Kl | 0.017 |
| H3BO3 | 0.006 |
| Hydrocortisone | 0.180 |
| Putrescine•2HCl | 5.000 |
| linoleic acid | 0.097 |
| thioctic acid | 0.239 |
| D-glucose (Dextrose) | 10072.0 |
| PVA | 2560.0 |
| Nucellin | 20.000 |
| Sodium Pyruvate | 18.392 |

After continuous culture in these mediums, the cell lines were evaluated in a fed-batch production assay, in which cells were evaluated in production medium B, illustrated in FIG. 2b. The cells were cultured at 37° C. for four days and then the temperature was shifted to 31° C. until day 12, when the assay was complete. The results indicate that cell lines that over express recombinant protein may also be adapted under production-matched conditions to achieve superior growth characteristics in a fed-batch production assay.

### Example 3

### Adaptation of Untransfected Chinese Hamster Ovary Cells to Low Insulin Conditions

Insulin directly impacts the metabolism of glucose by mammalian cells. The rapid consumption of glucose in cell culture is frequently coupled with the excretion of lactic acid as a metabolic waste product. Lactic acid can inhibit cell growth and have negative effects on cell viability. Insulin is also reported to be growth factor for mammalian cells.

Cells from the CHOK1 host cell line were taken from normal growth media (containing 10 mg/L nucellin) and put into media completely lacking insulin. After an initial lag phase in which the cells demonstrated diminished growth, the growth rate eventually climbed to a rate comparable to that of the CHOK1 control culture in insulin containing media, suggesting that cells cultured in the absence of insulin have adapted to these conditions. The viability of the cells was not affected and remained in the mid-90s throughout adaptation. This adaptation was continued for approximately 100 population doublings. When the adapted CHOK1-insulin cells were banked and then thawed, they maintained their ability to grow in insulin-free media. CHOK1 control cells thawed into insulin-free media showed a reduction in growth rate suggesting that the K1-insulin adaptation indeed altered the cells, allowing them to grow independent of exogenous insulin. Continuing investigations include combining the insulin-free phenotype with the 7-day passage adaptation phenotype.

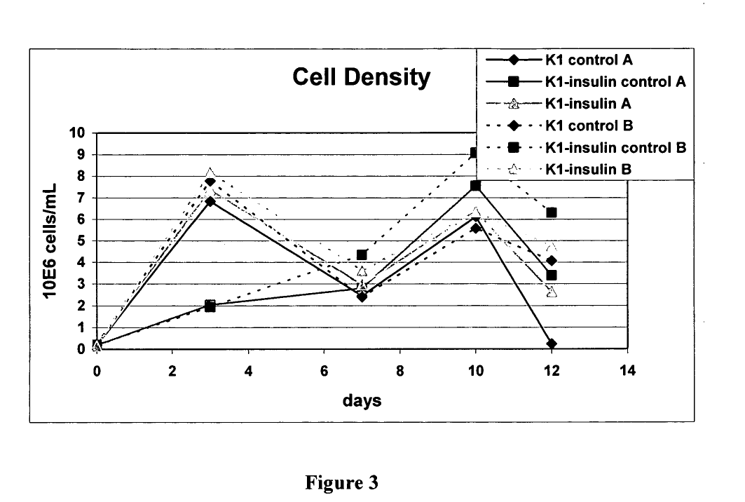

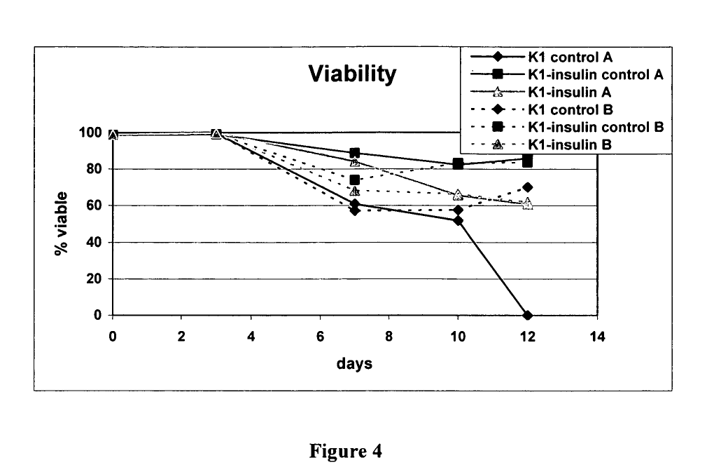

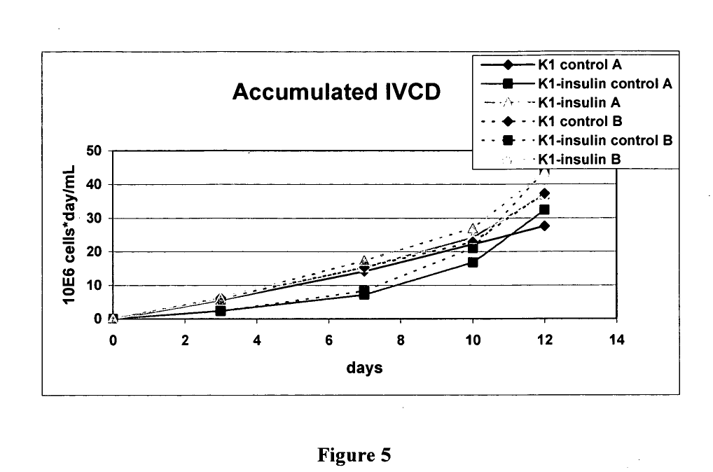

The first fed-batch production assay was set up in a non-pH adjusted format. The experiment included non-adapted CHOK1 cells in standard production media (20 mg/mL nucellin) as a positive control, and non-adapted CHOK1 cells cultured in insulin-free production media as a negative control. The insulin-free adapted CHOK1 cells were cultured in insulin-free production media. Insulin was included in the feed media on days 3 and 7 (at a final concentration of 0.006 mg/mL). Examination of the data suggests the CHOK1 cells adapted to grow in insulin-free media have very similar growth, viability and IVCD characteristics when compared to the CHOK1 positive control sample (see FIGS. 3-5). The negative control CHOK1 sample showed a reduction in growth rate (see FIG. 3). These results confirm that CHOK1 cells adapted to insulin-free growth are inherently different from the non-adapted CHOK1 cells, given their ability to grow well in media that does not contain insulin.

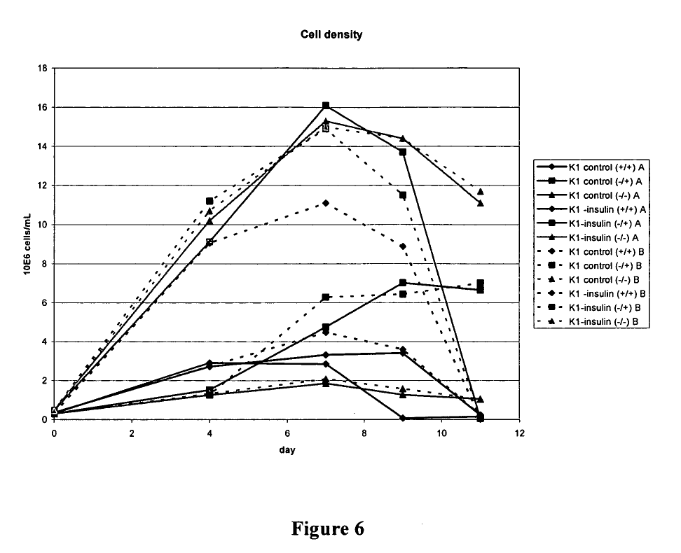

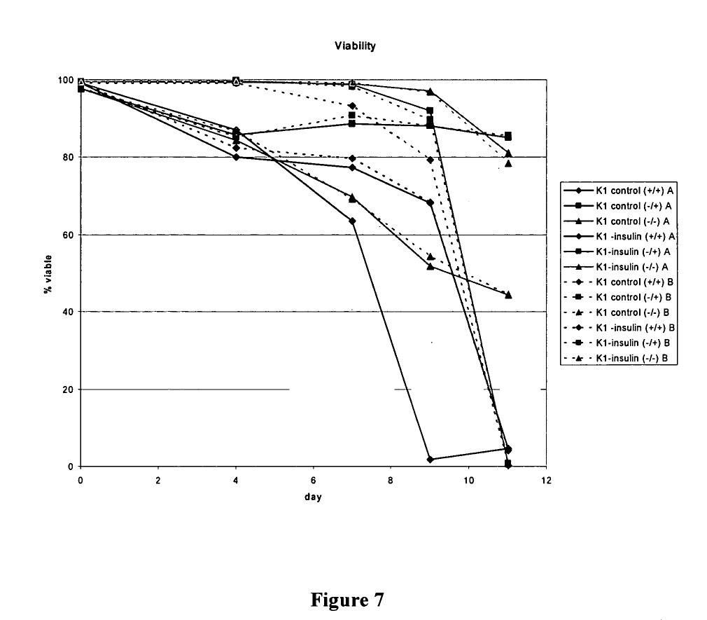

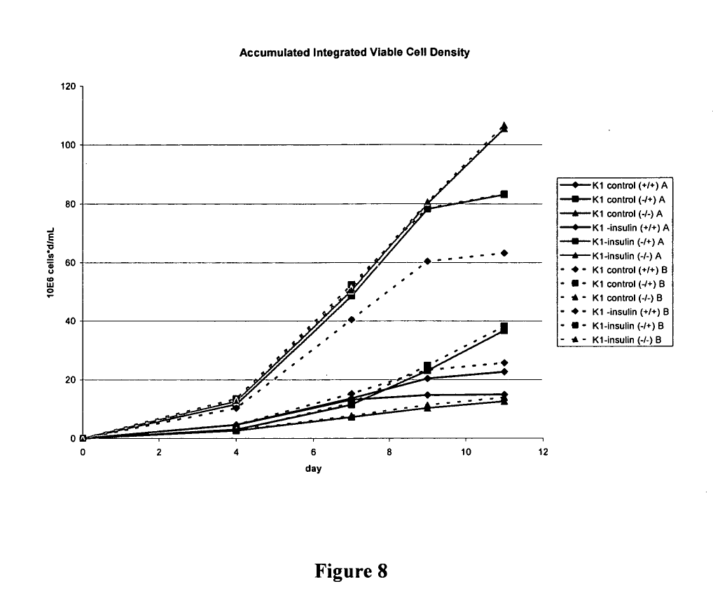

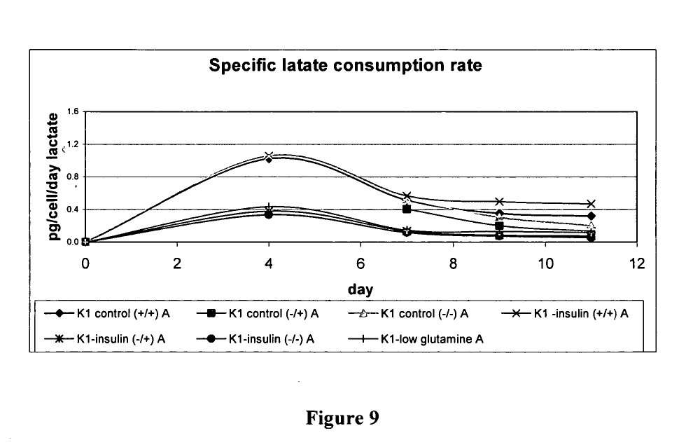

The insulin-free adapted CHOK1 cells were then evaluated in a second fed batch experiment, under conditions with pH control. In this experiment the non-adapted CHOK1 cells and insulin-free adapted CHOK1 cells were set up in three separate conditions. The first condition contained insulin in both the base media and the feed (50 mg/L and 0.006 mg/mL respectively), symbolized in FIGS. 6-9 as (+/+). The second condition contained insulin-free base media with an insulin containing feed (symbolized by (−/+) in FIGS. 6-9) and the third condition contained insulin-free base media and insulin-free feed media (symbolized by (−/−) in FIGS. 6-9). The insulin-free adapted CHOK1 cells demonstrate better growth, viability and IVCD when cultured in insulin-free media as compared to the non-adapted CHOK1 cells (see FIGS. 6-9). The CHOK1-insulin adapted cell lines seem to produce less lactate and almost completely consume whatever lactate they do produce (see FIG. 9). This elimination of a detrimental byproduct leads to a healthier cell culture and is seen as a very promising phenotype. This promising phenotype provides better growth conditions and allows the cells to reach higher densities then the associated control. It should be noted that this phenotype is directly related to the elimination of insulin from the media, as the CHOK1-insulin adapted cell lines has similar lactate production rates to the control sample when cultured in media containing insulin.

Example 3 demonstrates that adaptation of CHOK1 cells to an insulin-free conditions leads to desirable phenotypes when cells are cultured in industrially relevant production modes. The improved metabolic phenotypes lead to increased cell growth and viability, which are expected to have a significant positive impact on the volumetric productivity of a recombinant CHO cell culture.

Although certain embodiments of the disclosure have been described herein, the above description is merely illustrative. Further modification of the embodiments herein disclosed will occur to those skilled in the cell culture art and all such modifications are deemed to be within the scope of the embodiments as defined by the appended claims.

1. A method for adapting cells to protein production conditions comprising:
culturing the cells in an adaptation medium;
screening the cells and selecting a subpopulation of cells that exhibits an improved cell culture characteristic when the subpopulation is grown under protein production conditions, which characteristic differs from a corresponding cell culture characteristic that would be observed in cells grown in a medium that is not an adaptation medium, wherein the improved cell culture characteristic is selected from the group consisting of: improved growth, increased viability, increased integrated viable cell density, increased titer, increased cell specific productivity, and combinations thereof.

2. The method of claim 1, further comprising the step of passaging the cells prior to the screening step.

3. The method of claim 2, wherein the step of passaging comprises passaging the cells two or more times prior to the screening step.

4. The method of claim 2, wherein the step of passaging comprises passaging the cells after approximately 3 or 4 days in the adaptation medium.

5. The method of claim 2, wherein the step of passaging comprises passaging the cells after approximately 7 or 8 days in the adaptation medium.

6. The method of claim 2, further comprising the step culturing the cells in a medium that is not an adaptation medium after the passaging step.

7. The method of claim 6, wherein the medium that is not an adaptation medium is a standard growth medium.

8. The method of claim 1, wherein the cells are not transfected.

9. The method of claim 1, wherein the cells have been transfected to express a protein of interest.

10. The method of claim 9, wherein the protein of interest is an antibody.

11. The method of claim 9, wherein the protein of interest is a protein therapeutic.

12. The method of claim 1, wherein the adaptation medium comprises a production medium.

13. The method of claim 12, wherein the production medium comprises an increased level of one or more medium components as compared to a standard growth medium, the medium components selected from the group consisting of: nutrients, vitamins, trace elements, and combinations thereof.

14. The method of claim 1, wherein the adaptation medium comprises a secondary metabolite selected from the group consisting of: lactate, ammonia, and combinations thereof.

15. The method of claim 14, wherein the secondary metabolite is added to the adaptation medium at the beginning of the cell culture.

16. The method of claim 14, wherein the level of the secondary metabolite is increased as the cell culture progresses.

17. The method of claim 16, wherein the level of the secondary metabolite increases due to metabolic activity of the cells.

18. The method of claim 16, wherein the level of the secondary metabolite increases through addition of the secondary metabolite to the cell culture.

19. The method of claim 14, wherein the selected subpopulation of cells take a secondary metabolite, such that the level of the metabolite decreases when the adapted cells are grown in a production medium.

20. The method of claim 1, wherein the adaptation medium comprises one or more inhibitors selected from the group consisting of: lactate, ammonia, alanine, glutamine, acetolactate, and combinations thereof.

21. The method of claim 14 or 20, wherein lactate is present in a concentration of about 2 to about 10 g/L.

22. The method of claim 21, wherein lactate is present at the beginning of the cell culture in a concentration of about 2 to about 10 g/L.

23. The method of claim 14 or 20, wherein ammonia is present in a concentration of about 0.1 to about 0.5 g/L.

24. The method of claim 23, wherein ammonia is present at the beginning of the cell culture in a concentration of about 0.1 to about 0.5 g/L.

25. The method of claim 1, wherein the adaptation medium lacks insulin.

26. The method of claim 1, wherein the adaptation medium includes insulin at a concentration lower than about 10 mg/L.

27. The method of claim 1, wherein the cells are mammalian.

28. The method of claim 1, wherein the protein production conditions comprise conditions used in a bioreactor.

29. The method of claim 28, wherein the bioreactor is a production bioreactor.

30. The method of claim 1, wherein the protein production conditions comprise culturing the cells in a fed-batch protein production process.
